(19) United States
(12) Patent Application Publication
(10) Pub. No.: US 2025/0001170 A1
(43) Pub. Date: Jan. 2, 2025

(54) DEVICE FOR CARYING OUT DIATHERMIC AND MUSCLE STIMULATION TREATMENTS

(71) Applicant: Brera Medical Technologies S.R.L, Ogliastro Cilento (SA) (IT)

(72) Inventor: Attilio Grattacaso, Ogliastro Cilento (SA) (IT)

(73) Assignee: Brera Medical Te, Ogliastro Cilento (SA) (IT)

(21) Appl. No.: 18/576,175

(22) PCT Filed: Jun. 1, 2022
(86) PCT No.: PCT/IB2022/055116

§ 371 Date: Jan. 3, 2024

(30) Foreign Application Priority Data
Jul. 14, 2021 (IT) 102021000018503

(51) Int. Cl. A61N 1/36; A61N 1/04

(52) U.S. Cl. CPC A61N 1/36003; A61N 1/0452; A61N 1/0476; A61N 1/36034

(57) ABSTRACT

A device for diathermic and muscle stimulation treatments, comprising: a power generator, to which there are connected a return electrode, and at the opposite pole, at least two electrodes positioned on a handpiece, an electronic control unit configured to generate a voltage signal between the at least two electrodes on the handpiece and the return electrode, said signal being made up of the combination of: a first functional signal comprising a series of pulses in the frequency spectrum generating diathermy; a second functional signal comprising a series pulses for which the envelope of the maximum voltage provides a waveform equal to the action potential of muscle, said first and second signal being combined so that their amplitude is controlled in independent way.

### BACKGROUND OF THE INVENTION

### 1. Field of the Invention

The present invention relates the technical field comprising devices for carrying out treatments on tissues and muscles, in particular diathermy and electrostimulation treatments.

Diathermy is a therapy consisting in increasing temperature of an inner body part by applying low-voltage and high-voltage current, magnetic waves and ultrasound thereto; it is used for the treatment of some rheumatic muscle diseases. Instead, muscle electro- stimulators are made up of a low frequency electric current generator connected to a series of electrodes, and their usage is indicated to develop and strengthen muscles. The present invention relates to a device for treatments electro-stimulated by combined action of diathermy and muscle stimulation. More in particular, the present invention provides a device able to treat muscles by applying heat developed by diathermy combined with muscle electrostimulation.

### 2. Brief Description of the Prior Art

Many kinds of devices for carrying out diathermy are known and commonly used at the state of the art, among which those described in documents US2017189227, WO2013103891, U.S. Pat. No. 4,249,437, ITNA20110019, U.S. Pat. No. 7,035,691. Generally, these electromedical machines are operated by applying two electrodes, for example by using adhesive plates on the body area to be treated; in the following currents with intensity chosen according to the patient pathology are applied to the patient by means of said electrodes.

The currents applied develop, by Joule effect, an amount of heat in the patient's body, which is a function of the body impedance and so depends on the distance between the electrodes arranged on the body area to be treated.

The heat developed in this way will cause an increase in temperature, not superficial but localized in the deep layers, in the body area of the patient in the path between the two electrodes. The reached temperature is controlled by variation of the applied power and can be chosen according to the pathology to be treated.

Such treatment is very much used in sport rehabilitation, and it is often followed by an electrostimulation treatment in order to increase its effectiveness.

In fact, the body heating stimulates lipolysis, i.e. the metabolic process which provides triglycerides cleavage in free fatty acids and glycerol. These fatty acids are then consumed thanks to the muscle electrostimulation treatment, whose effectiveness is maximized by the previous diathermic treatment, since the duration of the electrostimulation treatment being equal, a heated muscle burns more fatty acids than a not heated muscle.

Today, diathermy is used in sport rehabilitation as preparatory step to electrostimulation in order to maximize its effects.

Combining diathermy with electrostimulation would be very convenient, in fact it would allow to treat each muscle faster than by the only electro-stimulating effect. The treatment time reduction would allow obviously to treat more muscle groups in only one session, or, as an alternative to be able to treat more patients at the same time.

There exist devices combining the heat application with the electrostimulation in many ways.

For example, microwaves for heat generation are used together with the electrostimulation for muscle stimulation. Anyway, since microwaves are high frequency electromagnetic fields, there exists a risk linked to their usage both for operator and patient.

It is known in fact that the more the irradiation frequency increases, the more the interaction with the surrounding biological tissues is; moreover, microwaves have reduced penetration depth in tissues, since the most energy part is absorbed by the superficial layers.

There exist also devices able to carry out diathermy and electrostimulation, as for example US2021/0023364A1, which are however characterized by the need to use more discrete generators for each energy source.

Disadvantageously, these machines are provided with discrete couples of electrodes for each energy source and also use DC pulses for muscle stimulation (EMS), which, as known in literature, have clear limits of action range due to the high impedance of the superficial tissues at such frequencies.

Another limit of these devices is the fact that they are provided with a flat electrode surface, and so during the passage of the electrode on the concave and convex areas of the skin surface, a large part of the electrode surface loses contact with the skin.

In this case it occurs that, the irradiation power being equal, the energy density for surface unit of the electrode is greater, and so there occur overheating phenomena of the treated area with risk of burns. To avoid burns due to this problem, in the devices known at the state of the art, it is needed to use a lower electric intensity of the diathermic treatment than the one really needed. But, in this way, the heating application is mild and as a consequence the effectiveness of the treatment is remarkably reduced.

Another limit of the devices known at the state of the art is that the handpiece on which the electrode is installed which slides on the skin, since it is moved by an operator, has necessarily a not constant sliding speed on the body surface.

If such speed variation is very marked, since electrostimulation and diathermy parameters do not depend on the handpiece acceleration, it occurs that in the areas where speed is higher, the heat and electrostimulation application is lower, and vice versa.

Clearly, such aspect causes a lower treatment effectiveness in those areas where abrupt speed variations occur, and the treatment will depend substantially on the operator's skill in keeping the sliding speed constant without abrupt accelerations during the passage on the skin.

There exist also many models of muscle electro stimulators which allow to maximize the treatment effectiveness.

An example is shown in U.S. Pat. No. 4,249,537A, which describes an apparatus to stimulate and control muscle contraction, which comprises conductive electrodes to contact positions at distance on the body. Another example is described in U.S. Pat. No. 7,035,691B2, which shows a method and device generating in the muscle resonant sequences having pulses at optimal intervals and configured to provide uniform contractions.

There exist also other known solutions which represent the usage of an electro- stimulator inside a temperature-controlled environment, in order to increase the effectiveness of the electrostimulation by means of heat application from outside.

### SUMMARY OF THE INVENTION

According to a first aim, the present invention provides a mono-source device configured to electro-stimulate the muscle in a deep and combined way with a diathermic treatment.

According to another aim, the present invention provides a device configured to maximize the diathermic heat application while avoiding burns of the treated area, in order to obtain an effective vasodilation effect which increases muscle oxygenation and maximizes treatment effectiveness. According to another aim, the present invention provides a device configured to adapt the electric intensity parameters of the diathermic treatment and electrostimulation, while compensating the speed variations during the electrode sliding on the area to be treated.

According to another aim, the present invention provides a device configured to vary intensity and muscle heating depth so to be able to carry out a combined and optimized action of electrostimulation in independent way.

The present invention realizes the prefixed aims since it is a device for carrying out diathermic and muscle stimulation treatments, comprising:
a single power generator (1), to which there are connected a return electrode (2), and at the opposite pole, at least two electrodes (3), positioned on a handpiece (10) and separated from each other by means of insulating material (9), an electronic control unit (4) configured to manage the power supplied by said power generator (1) between said return electrode (2) and said at least two electrodes (3), characterized in that said electronic control unit (4) is configured to generate a whole voltage signal, made up of the combination of two functional signals, between the electrodes on the handpiece (3) and the return electrode (2);
a first functional signal comprising a series of pulses (7) in the frequency spectrum generating diathermy;
a second functional signal comprising a series of time variable amplitude radiofrequency pulses, for which the development of the maximum voltage of the pulse over time provides a waveform equal to the development of the action potential of muscle (8) able to overcome the skin resistance and to reach the deep layers,
   said first and second signal being combined so that the amplitude thereof is controlled in independent way.

### BRIEF DESCRIPTION OF THE DRAWINGS

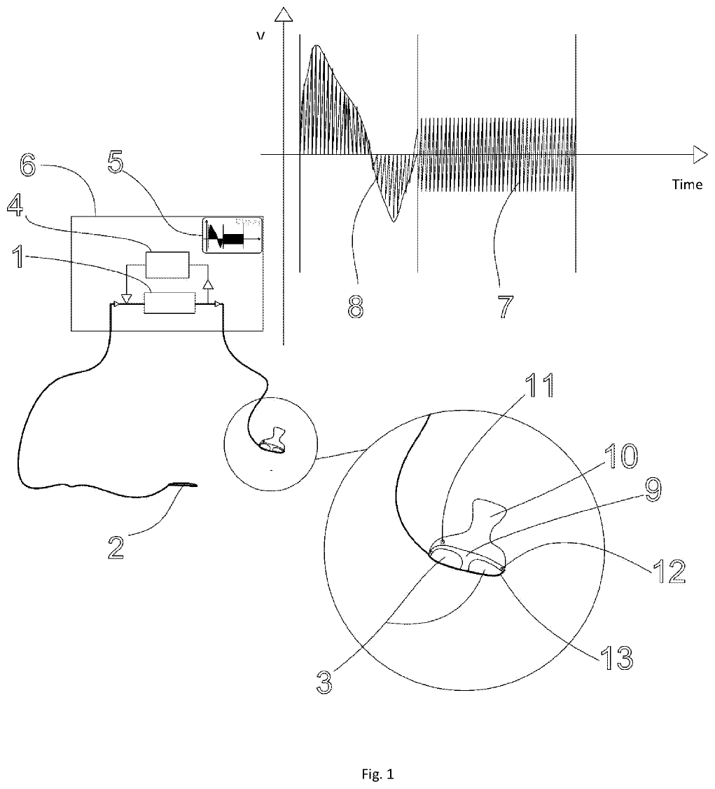

The device will be now described with reference to the appended FIGS. 1 to 6.

In FIG. 1, it is shown a schematic view of the main elements of the device;

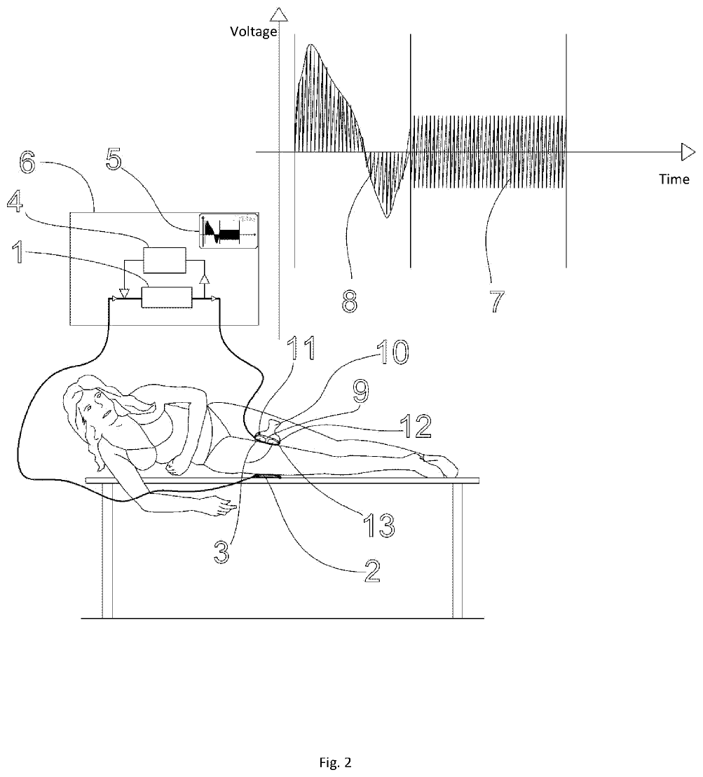

in FIG. 2, it is shown a treatment example by means of the device;

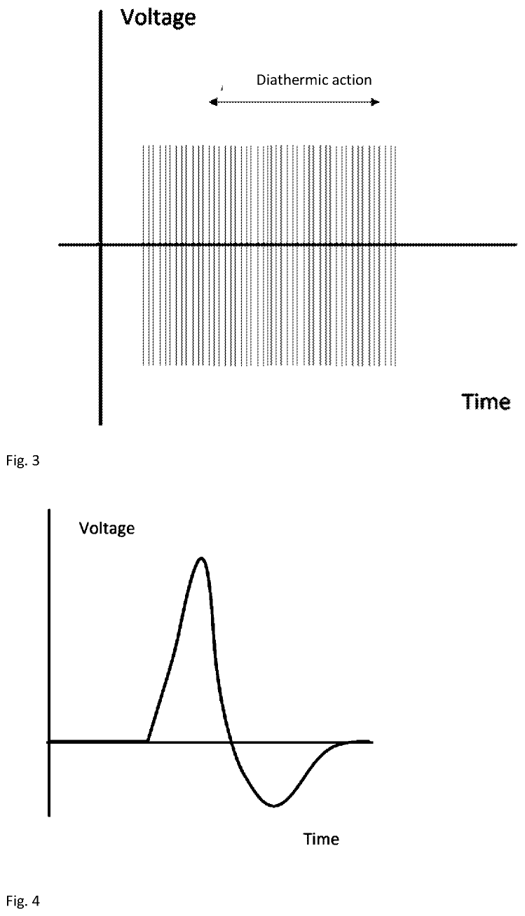

in FIG. 3, on a time/applied voltage graph it is shown an example scheme of the signal used to carry out a diathermic treatment and whose values of applied voltage have constant peaks and null average value;

in FIG. 4, it is shown the development of the time of muscle action potential;

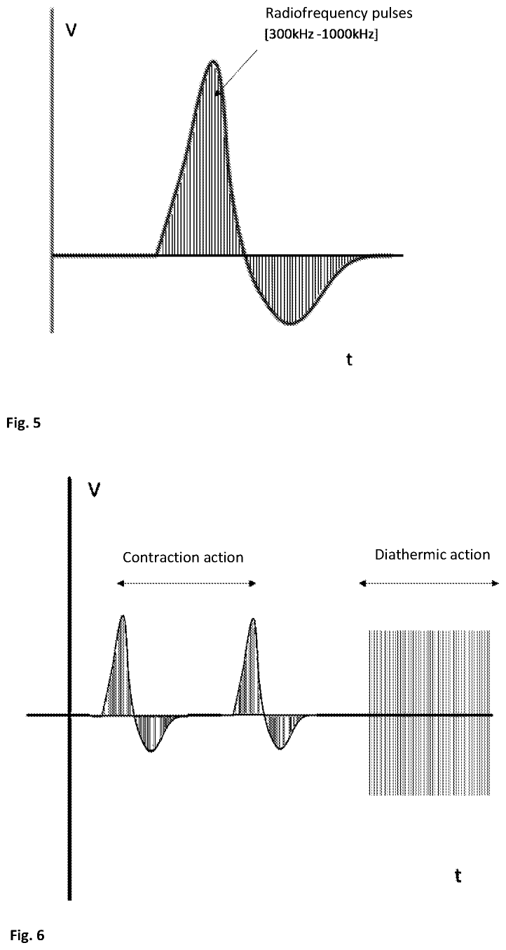

in FIG. 5, it is shown an action potential pulse obtained by the modulation of the radiofrequency portion,

in FIG. 6, it is shown a combination example of the two above-described functional signals which produce a contraction effect and a diathermic effect in tissues.

### DETAILED DESCRIPTION OF THE PREFERRED EMBODIMENTS

With reference to what shown in the appended figures, in a preferred embodiment the device according to the invention comprises:
a handpiece (10) provided with at least two electrodes (3) separated by insulating material (9) from each other;
a single power generator (1) (shown inside a housing (6)), to which there are connected a return electrode (2) and, at the opposite pole, said at least two electrodes (3).

The device is configured so that the first electrode (2) can be positioned at contact with the skin at a muscle to be subjected to electrostimulation combined with diathermy, while said at least two electrodes (3) are positioned, by means of said handpiece (10), on the skin at the same muscle. The movement of the handpiece allows to carry out also a massage on such muscle.

The device comprises also an electronic control unit (4), electrically connected to the power generator (1) and configured to manage the power supplied by the power generator (1) between the first electrode (2) and said at least two electrodes (3).

The electronic control unit (4) is configured to generate a whole voltage signal, made up of the combination of two functional signals, between the electrodes on the handpiece (3) and the return electrode (2);
a first functional signal comprising a series of pulses (7) in the frequency spectrum generating diathermy;
a second functional signal comprising a series of time variable amplitude radiofrequency pulses, for which the development of the maximum voltage of the pulse over time provides a waveform equal to the development of the action potential of muscle (8), said first and second signal being combined so that the amplitude thereof is controlled in independent way.

It is to be specified that, with reference to the first signal, for frequency spectrum generating diathermy is intended the frequencies between 300 KHz and 1000 KHz. Moreover, preferably the average value of the signal is equal to zero, and the diathermic effect can be controlled by means of the signal amplitude (maximum voltage value).

It is to be specified instead, with reference to the second functional signal, that the repetition of this RF burst packet induces muscle contraction and the frequency thereof depends on the repetition over time of such packets.

The parameters which can be set are:
the duration of each functional signal can vary between 20 us and 40 us;
the repetition frequency of the second functional signal is between 0.5 Hz and 500 Hz;
the pulses have frequency between 300 KHz and 1 MHz.

In the time intervals between one muscle contraction and the other one a diathermic signal can be sent, of the kind schematized in FIG. 3, and corresponding only to the first functional signal previously described.

Furthermore, the device comprises means for measuring the current flowing through each of said at least two electrodes (3) positioned on the handpiece, and said control unit (4) is configured to reduce the intensity of the diathermic treatment, by reducing the voltage of said first functional signal in a directly proportional way to the number of at least electrodes provided on the handpiece (3) for which the measured current intensity is null.

Thanks to this invention, it is possible to control the stimulation pulse independently of the diathermic one starting from a single power generator, in fact both the control of the electro-stimulating pulse amplitude and of the diathermic action are extended. For example, by increasing the contraction pulse amplitude the contraction intensity is increased. By increasing the radiofrequency amplitude, the heat intensity transferred to the deep tissue is increased.

Moreover, preferably, to solve the technical problem of the abrupt speed variations linked to the not uniform sliding of the handpiece on the skin, and to make the treatment not operator-dependent the handpiece (10) can be provided with at least an accelerometer (11) connected to the electronic control unit (4). The electronic control unit is configured to receive in input an acceleration signal detected by said accelerometer, to integrate such signal to obtain a speed signal and to increase the power supplied in a directly proportional way to the sliding speed of the handpiece on the treated area.

In this way, the heat application is maximized as a function of the sliding speed of the handpiece on the skin and the treatment does not depend on the operator's skill in keeping the sliding speed constant, but it is adapted to the treatment automatically.

At the insulating area (9) dividing the at least two electrodes (3) it is preferably provided at least a temperature sensor (12), configured to detect the skin temperature value and to intervene by controlling and adapting the power and by interrupting the treatment immediately in case the skin temperature exceeds a predetermined alarm threshold. Moreover, preferably, the surface of the handpiece (10), on which said at least two electrodes (3) are positioned, is provided with a curved edge in contact with the skin (13) so to be adapted to the concave and convex surfaces of the body. Thanks to the heat application due to the diathermic effect, there occurs a vasodilation, the muscle fibers slide with less friction and at the same time there is a greater oxygenation of the muscle with the consequent reduction of lactic acid formation. By using a traditional electro stimulator, the muscle works anaerobically, this fact contributes to the formation of lactic acid which causes pain, and so it is needed to reduce the muscle working cycle with a typical ratio 1 to 5. As a consequence, it is normally used a lower stimulation with a reduced effect on the muscle. Moreover, by using a traditional electro stimulator, which uses lower frequencies, the deeper muscle fasciae are not interested but only the surface is, on the contrary the generated contraction pulse, since it is a radiofrequency pulse, is able to interest the deep muscle fasciae since the tissue impedance is lower at such frequencies. Moreover, by varying the frequency of the functional signal (7) it is possible to act on the muscle heating depth, and so to carry out a combined and optimized action of electrostimulation, by acting instead on the amplitude and duration it is possible to manage the intensity of the action potential of the muscle (8) and so on the contraction intensity of the same muscle.

In a preferred embodiment, at least one of the electrode provided on the handpiece acts as return electrode.

In another preferred but not limiting embodiment, the device comprises a plurality of handpieces and a plurality of power generators (1), configured each to control a respective handpiece, so that a plurality of handpieces can function simultaneously.

1. A device for carrying out diathermic and muscle stimulation treatments, comprising:
a single power generator (1), to which there are connected a return electrode (2), and at the opposite pole, at least two electrodes (3), positioned on a handpiece (10) and separated from each other by means of insulating material (9), an electronic control unit (4) configured to manage the power supplied by said power generator (1) between said return electrode (2) and said at least two electrodes (3),
and wherein said electronic control unit (4) is configured to generate a whole voltage signal, made up of the combination of two functional signals, between the electrodes on the handpiece (3) and the return electrode (2);
a first functional signal comprising a series of pulses (7) in the frequency spectrum generating diathermy;
a second functional signal comprising a series of time variable amplitude radiofrequency pulses, for which the development of the maximum voltage of the pulse over time provides a waveform equal to the development of the action potential of muscle (8), said first and second signal being combined so that the amplitude thereof is controlled in independent way.

2. The device according to claim 1, wherein said first signal has a null average value and frequency between 300 kHz and 1000 MHz.

3. The device according to claim 1, wherein said second signal is characterized by:
a duration of the waveform between 20 us and 40 us;
a repetition frequency between 0.5 Hz and 500 Hz;
and in that said radiofrequency pulses have frequency between 300 kHz and 1 MHz.

4. The device according to claim 1, wherein said whole voltage signal comprises a cyclic repetition of a signal deriving from the combination of said two functional signals.

5. The device according to claim 1, further comprising means for measuring the current flowing through each of said at least two electrodes (3) positioned on the handpiece.

6. The device according claim 1, wherein said control unit (4) is configured to adapt the intensity of the diathermic treatment, by varying the voltage of said first functional signal in a directly proportional way to the number of electrodes provided on the handpiece (3) for which the measured current intensity is null.

7. The device according to claim 1, further comprising at least one accelerometer (11) integral with said handpiece (10) and connected to said electronic control unit (4), said electronic control unit being configured to receive in input the acceleration signal detected by said accelerometer, to integrate such signal to obtain a speed signal and to adjust the power supplied in a directly proportional way to the sliding speed of the handpiece on the treated area.

8. The device according to claims 7, wherein said handpiece (10) comprises also at least a temperature sensor (12) configured to detect the skin temperature value.

9. The device according to claim 8, wherein the surface of the handpiece (10), on which said at least two electrodes (3) are positioned, is provided with a curved edge (13) in contact with the skin.

10. The device according to claim 9, wherein at least one of the electrodes provided on the handpiece acts as return electrode.

11. The device according to claim 1, further comprising a plurality of handpieces and a plurality of power generators (1), configured each to control a respective handpiece, so that a plurality of handpieces can function simultaneously.
